(19) United States
(12) Patent Application Publication
(10) Pub. No.: US 2017/0100190 A1
(43) Pub. Date: Apr. 13, 2017

(54) HIGH PERFORMANCE MATERIAL FOR ELECTRO-SURGICAL VAPORIZATION ELECTRODES

(71) Applicant: Mysore Wifiltronics PVT LTD, Mysore (IN)

(72) Inventor: Alok Gupta, Mysore (IN)

(21) Appl. No.: 15/077,259

(22) Filed: Mar. 22, 2016

(30) Foreign Application Priority Data
Oct. 12, 2015 (IN) 5444/CHE/2015

(51) Int. Cl. A61B 18/14

(52) U.S. Cl. CPC A61B 18/1492; A61B 2018/1417; A61B 2018/1407; A61B 2018/00625

(57) ABSTRACT

Surgical resection electrodes use a tungsten heavy metal alloy for construction of the active head, providing long operating time as compared to conventional designs. A bore-through design adds to the stability of the electrode head and provides full electrode ignition very efficiently. Angled branch wire connections enhance visualization of tissues and minimize deterioration of insulation by reducing buildup of concentrated heat. A cavity on the back side of the electrode blocks any discharge from leads' insulation at the back side, thus reducing the risk of premature breaking of the lead wires because of sparking.

### BACKGROUND OF THE INVENTION

Field of the Invention

The present invention generally relates to electrosurgical vaporization electrodes which are used in endoscopic surgeries in urology, gynecology and gastroenterology. In particular, it relates to the material used for manufacturing the distal ends of the vaporization electrodes to enable more tissue removal before the necessity of replacing the electrode. Such electrodes are also commonly described as monopolar or bipolar resection electrodes.

Description of the Related Art

Endoscopic electrosurgery for removal of unwanted tissue is well known in the surgical field. Transurethral resection of the prostate (TURP) in urology, endometrial fibroid ablation in gynecology, and polyp resection in gastroenterology are examples of such electro surgeries.

Original electrosurgical systems are monopolar in design where the electrodes carry the active charge of radio frequency and the return path is through the patient body, attaching a grounding pad to the patient's thigh. Such systems and electrodes are described in U.S. Pat. Nos. 2,448,741, 2,888,017, 3,752,159, 3,856,015, 3,973,578 and 4,134,406. Most of these electrodes use wire elements in U-shaped loop form for scooping out the unwanted tissue. U.S. Pat. Nos. 5,549,605, 5,599,349 5,669,906, 5,766,168, 5,908,419 and 6,197,025 describe the next generation of electrodes which have many different designs of the distal heads in the shape of rollers, grooved rollers, V-rollers, sliders, grooved sliders and wires in different shapes where the electrodes are used for coagulating and vaporizing, apart from just scything and scooping the tissue.

These electrodes are used inside endoscopes comprised of telescope, fiber light, irrigation system and working element to manipulate the electrodes. Such endoscopic systems are well described in U.S. Pat. Nos. 3,835,842, 4,149,538, 4,955,884, 5,112,329, 5,151,101 and 5,423,813.

Radio frequency (RF) power is transferred to the active distal heads of electrodes at different power settings for cutting, coagulating and vaporization. Such electrical generators with proper safeguards are described in U.S. Pat. Nos. 6,093,186 and 7,211,081.

Monopolar resection is carried out using non-conductive irrigation solution so that the RF energy passes from the electrode to the tissue only where the electrode element comes in contact with the tissue. Such solution normally is Sorbitol, which gives other complications in human body when absorbed during prolonged surgery.

This problem has been addressed by using a bipolar resection system where normal saline solution is used for surgery. This system is well described in U.S. Pat. Nos. 3,901,242; 4,060,087 and 6,113,597.

In recent practice of bipolar saline surgeries, vaporization of tissue rather than cutting or scooping is becoming quite popular. Such hemispherical shaped electrodes, also known as button or mushroom electrodes, are described in U.S. Pat. Nos. 8,265,727 and 2014/0,378,965. Vaporization electrodes have the advantage of coagulating the bleeding vessels while vaporization is occurring.

Electrode heads for cutting applications are made of tungsten wire formed in a U-shape so that unwanted tissue can be removed by scooping when the heated electrode is moved in forward or reverse direction using the mechanism in the working element of the resectoscope. For coagulating the larger bleeding vessels, roller, ball and slider designs, with and without grooves, are used for electrode heads. For simultaneous evaporation and coagulation, hemispherical designs, also called button or mushroom, are used.

Currently stainless steel and brass material, bare or plated with gold, are used for manufacture of these vaporization heads, which are in the shape of rollers, grooved rollers, V-rollers, sliders, grooved sliders, balls, hemispheres, grooved hemispheres, buttons, mushroom, scythes, etc.

### SUMMARY OF THE INVENTION

The present invention is generally directed to a resection head comprising an electrode and an electrically conductive wire attached to the electrode. In some embodiments, the electrode is formed of a heavy metal alloy material. In particular the electrode can be constructed of a tungsten alloy material having a composition which includes at least two of nickel, copper or iron, with the majority comprising of tungsten. The electrode can have a bore hole formed therein, with a loop of the electrically conductive wire passing through the bore hole. In some embodiments, the electrode has a front side for engaging tissue to be vaporized with a cavity formed in the front side, wherein the cavity is in fluid communication with the bore hole. In some embodiments the electrode further has a planar back side generally opposite the front side with another cavity formed in the planar back side, and the electrically conductive wire has two wire sections leading to the planar back side with an insulating material surrounding the wire sections, the insulating material extending from each of the wire sections into the cavity and abutting the electrode. The two wire sections preferably form an angle in the range of 70° to 90°.

The above as well as additional objectives, features, and advantages in the various embodiments of the present invention will become apparent in the following detailed written description.

### BRIEF DESCRIPTION OF THE DRAWINGS

The present invention may be better understood, and its numerous objects, features, and advantages of its various embodiments made apparent to those skilled in the art by referencing the accompanying drawings.

FIG. 1a is a resection electrode assembly with a hemispherical vaporization electrode head (Hemi Electrode) constructed in accordance with one embodiment of the present invention;

FIGS. 1b and 1c are side and front sectional views of the hemispherical vaporization electrode head of FIG. 1a;

FIGS. 2a and 2b are perspective and side views of another hemispherical vaporization electrode head constructed in accordance with another embodiment of the present invention;

FIGS. 3 and 4 are front sectional views of slider vaporization electrode heads constructed in accordance with other embodiments of the present invention;

FIGS. 5 to 9 are roller bar vaporization electrode heads constructed in accordance with other embodiments of the present invention; and

FIGS. 10 to 13 are roller ball vaporization electrode heads constructed in accordance with other embodiments of the present invention.

The use of the same reference symbols in different drawings indicates similar or identical items.

### DESCRIPTION OF THE PREFERRED EMBODIMENT(S)

While vaporization of tissue has several advantages over previous electrosurgical cutting techniques, vaporization is a much slower process of tissue removal as compared to scooping, resulting in a longer time for the electrode head remaining active. This extended time, coupled with the fact that electrode head has to operate at a much higher temperature and energy level for vaporization, makes the head wear out much faster. If a large mass of tissue needs to be removed, the electrode may need to be replaced before finishing, making the time for surgery even longer. Wear out of these materials is quite high in monopolar applications and much higher in bipolar applications because of the presence of plasma temperatures and a corrosive environment. It would, therefore, be desirable to devise an improved vaporization electrode head that could better withstand these degradative conditions. It would be further advantageous if an improved vaporization electrode head could be designed to provide more focused energy at the point of tissue removal. It would also be advantageous if an improved vaporization electrode head could be designed to give the operator greater visual access at the point of tissue removal.

These and other advantages are achieved in a vaporization electrode head which uses a high performance material to substantially reduce the wear of the head, both in monopolar and bipolar applications. Accordingly, the electrode head can be made from heavy metal alloys such as tungsten alloys comprising 85% to 96% by weight of tungsten and the balance of nickel, iron, cobalt or copper in differing proportions. The electrode head can also provide a bore-through design, adding to the stability of the electrode head and providing higher power density at the required point of operation by giving full electrode ignition in a very efficient manner. The electrode head can further use an angled design for the branch-wires, which enhances visualization of the target tissue and minimizes deterioration of wire insulation by minimizing the buildup of heat at the proximal end.

The electrosurgical vaporization device in accordance with the present invention will now be described with reference to the accompanying drawings, which do not limit the scope and ambit of the disclosure.

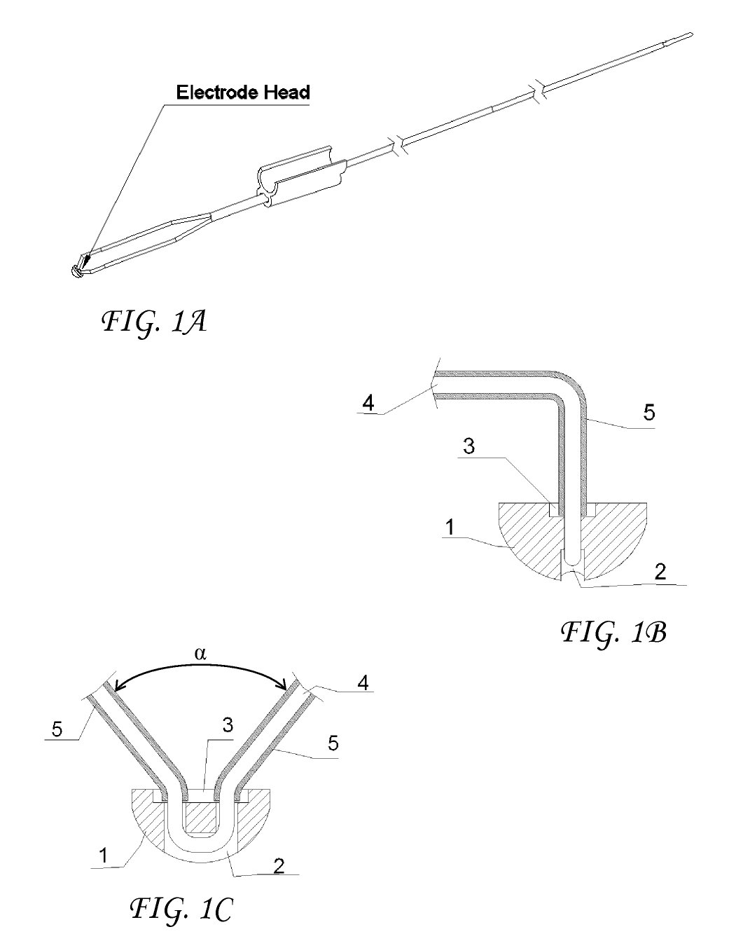

FIG. 1a shows an exemplary resection electrode assembly designed in accordance with the present invention which may be used for endoscopic electrosurgery in, e.g., urology, gynecology and gastroenterology. The assembly includes an electrically conductive wire having a proximal portion and a distal portion, with an electrode head located at the distal portion. The electrode head of the assembly can be made in different configurations.

FIGS. 1b and 1c show two views of one such embodiment for a hemispherical evaporation electrode as per the present invention. In these figures electrode head 1 is made from a tungsten alloy. A cavity 2 has been formed in the electrode head as a bore-through for passing tungsten wire lead 4 to transfer the RF energy. Sleeve 5 is an insulating material, specifically made of polytetrafluoroethylene (PTFE) in this embodiment. Another cavity 3 has been made on the planar back side of the electrode head in such a fashion that it allows the insulating sleeve 5 to butt against the electrode head, effectively blocking the tungsten wire lead from generating undesirable plasma at the back end of the electrode.

Electrode head 1, as shown in FIGS. 1b and 1c, is preferably made of a tungsten alloy, having the main constituent as tungsten and small quantities of other metals used as binders. An electrode head made from tungsten gives far longer life in resection operations for several reasons. During bipolar plasma operation, temperature in the vicinity of the head is very high. Tungsten, being a high temperature material, is able to give much longer life as compared to conventional stainless steel or brass heads. Bipolar resection carried out by creating plasma in a saline solution creates a very corrosive environment; once again tungsten, being very resistant to corrosion, is able to give a much longer life as compared to the conventional stainless steel or brass heads.

As shown in FIG. 1b, the bore-through design for the electrode head, with the wire leads extending from the branches, looping through the center of the hemispherical electrode firing surface provides many benefits. It adds to the stability of the electrode head. Power density is also higher due to having only to energize the looped wire that runs through the electrode head. Through the ignition of the single looped wire, full electrode ignition is possible in a very efficient way.

As shown in FIG. 1c, the electrode head can have angled branch wire connections, i.e., there is an angle α formed between the two branches of wires emanating from the electrode head as viewed from the front (or back). This construction provides two unique benefits. First, it enhances visualization of the target and surrounding tissues by creation of a V-shaped window. Second, it minimizes deterioration of insulation by reducing buildup of concentrated heat.

The angle can vary considerably depending upon application and preferences. The most desirable angle would be 180° (i.e., a straight line) for visual clarity, but this is not practical because of the limitations placed by the size of the endoscope instrument's outer sheath, which normally has outer diameter of 21 to 27 French (7-9 mm diameter). Different angles may be optimized for different space requirements. For 24 French design, the preferred angle is 80°; for 21 French it can be narrower (e.g., 70°), for 27 French it can be wider (e.g., 90°). The wire should be constructed of a material that is sufficiently rigid to maintain its geometry during use. High tensile strength tungsten wire can be used for this application, which is commercially available.

The specific composition of the alloy material of the electrode head can also vary. Generally, the electrode head is formed of a heavy metal alloy which, as used herein, includes pseudo alloys (pseudo alloys are blends which are formed of constituent materials at least some of which are not mutually soluble). Heavy metal alloys are those having a high density, preferably in the range of 17-19 g/cm³. Most preferably the heavy metal alloy is a pseudo alloy of tungsten with a nickel-iron or nickel-copper matrix, which can be produced by powder metal and sintering processes. Comparable densities can be reached by gold or platinum metals, but these are considerably more expensive. Other exotic materials like rhenium, uranium or tantalum achieve these densities, but are typically too difficult or expensive to obtain, or they have unsatisfactory mechanical and environmental characteristics.

Tungsten heavy metal alloys not only have high density but also exhibit excellent formability and machinability, outstanding corrosion resistance, high modulus of elasticity, impressive thermal conductivity and low thermal expansion. Some specific examples are given below with reference to “MT” identifiers commercially available from Midwest Tungsten Service of Willowbrook, Ill.

| | Name | | | | | |
|---|---|---|---|---|---|---|
| | MT- | | | | | |
| | 17C | MT-17F | MT-175 | MT-18C | MT-18F | MT-185 |
| Material | 90% W | 90% W | 92.5% W | 95% W | 95% W | 97% W |
| | 6% Ni | 7% Ni | 5.25% Ni | 3.5% Ni | 3.5% Ni | 2.1% Ni |
| | 4% Cu | 3% Fe | 2.25% Fe | 1.5% Cu | 1.5% Fe | 0.9% Fe |

As can be seen, the preferred additive constituents to the tungsten alloys are nickel, copper and iron. When used with different proportions, these constituents affect hardness, tensile strength, elongation, brittleness, coefficient of thermal expansion, thermal conductivity, corrosion resistance, electrical conductivity and magnetic permeability. Copper as a constituent will not be desirable for bipolar electrodes because of poor corrosion properties in plasma applications. Pure tungsten is not desirable because of its inherent brittleness.

Powders in the foregoing proportions can be sieve mixed and pressed in an NNS (near net shape) molding die using a hydraulic press to obtain 55%-65% of the theoretical density. These green pellets are sintered at 1200° C.-1400° C. in a hydrogen atmosphere furnace for 2-4 hours to obtain 92%-100% of the theoretical density. Holes and cavities can be hand cleaned, and final polishing can be performed in a barrel finishing machine using alumina media. The remainder of the electrode assembly can be constructed using conventional processes, tools and fixtures.

Different compositions of tungsten heavy metal alloys can be used for improving the performance of existing and future designs of vaporization electrodes. Apart from the press and sinter technique described above, other techniques, like metal injection molding (MIM), can also be used to fabricate such NNS parts. MIM is a metalworking process by which finely-powdered metal is mixed with a measured amount of binder material to comprise a feedstock capable of being handled by plastic processing equipment through a process known as injection mold forming. The molding process allows complex parts to be shaped in a single operation and in high volume. The process steps involve combining metal powders with wax and plastic binders to produce the feedstock mix that is injected as a liquid into a hollow mold using plastic injection molding machines. The “green part” is cooled and de-molded in the plastic molding machine. Next, a portion of the binder material is removed using solvent, thermal furnaces, catalytic process, or a combination of methods. The resulting fragile and porous part, in a condition called “brown” stage, requires the metal to be condensed in a furnace sintering process. MIM parts can be sintered at temperatures nearly high enough to melt the entire metal part outright (up to 1450° C.), at which the metal particle surfaces bind together to result in a final, 96%-99% solid density. The end-product MIM metal has comparable mechanical and physical properties with parts made using classic metalworking methods.

Other methods may also be used to fabricate the electrode heads in accordance with the present invention, such as micromachining tungsten heavy metal alloy stock bars.

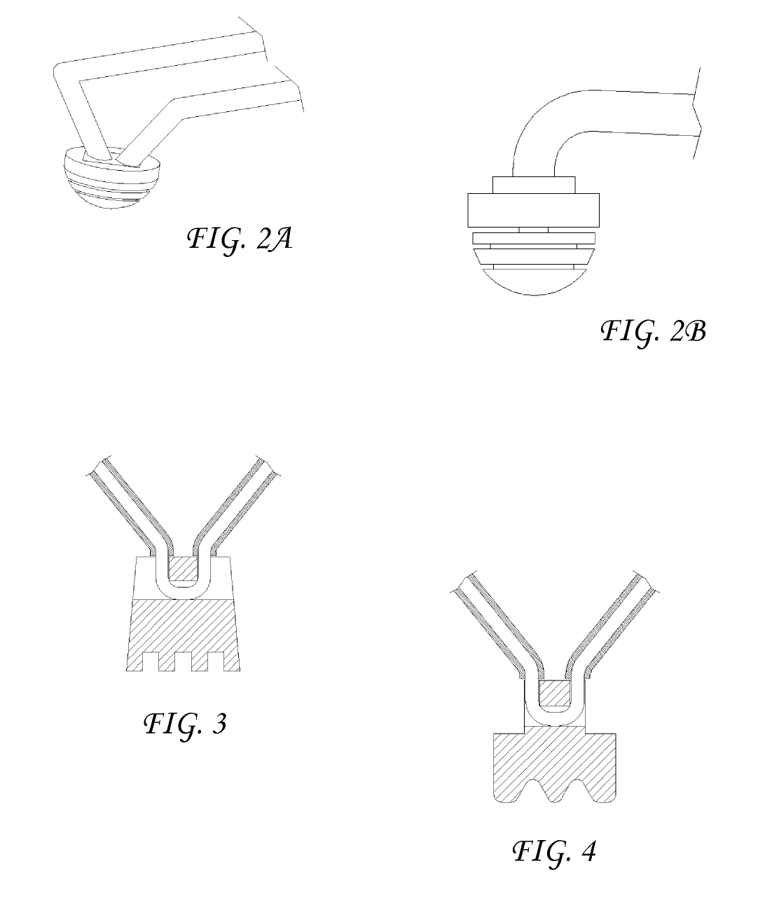

In simulated testing these tungsten alloy electrodes worked for a period exceeding conventional stainless steel electrodes by more than 50% in bipolar saline plasma environment, proving the superiority of this material. By corollary it can be stipulated that other vaporization electrode configurations, like roller bars, roller balls and sliders, made with tungsten heavy metal alloys will also perform better than the ones made with copper, brass or stainless steel. Some of such possible designs are illustrated in FIGS. 2 to 13 each of which can be constructed of the foregoing tungsten heavy metal alloys. FIGS. 2a and 2b are perspective and side views of a grooved hemispherical vaporization electrode head (Hemi Electrode). FIG. 3 is a square grooved slider electrode head.

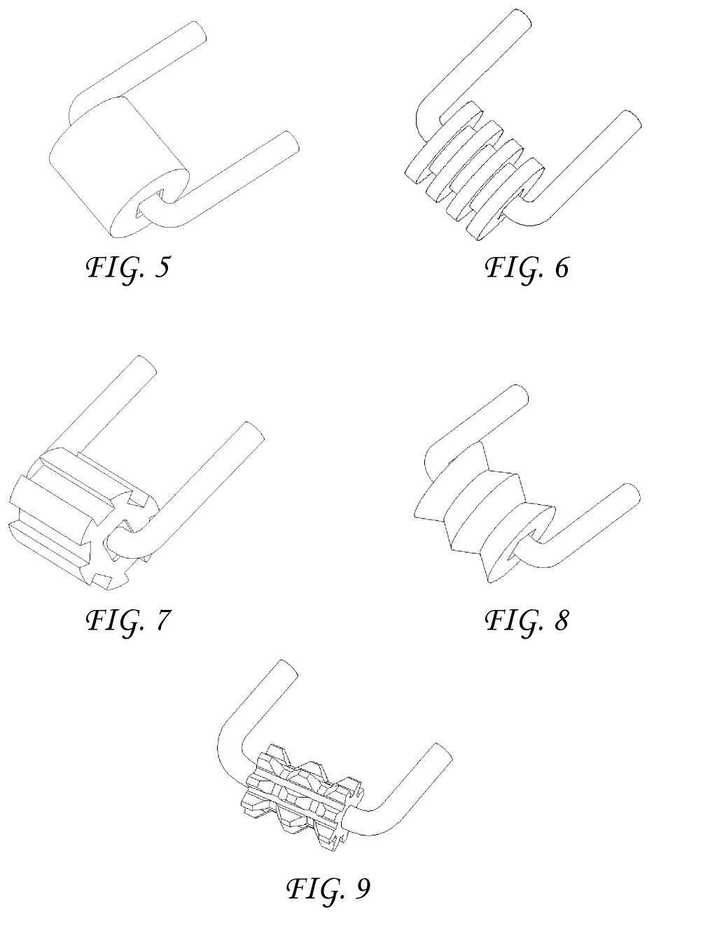

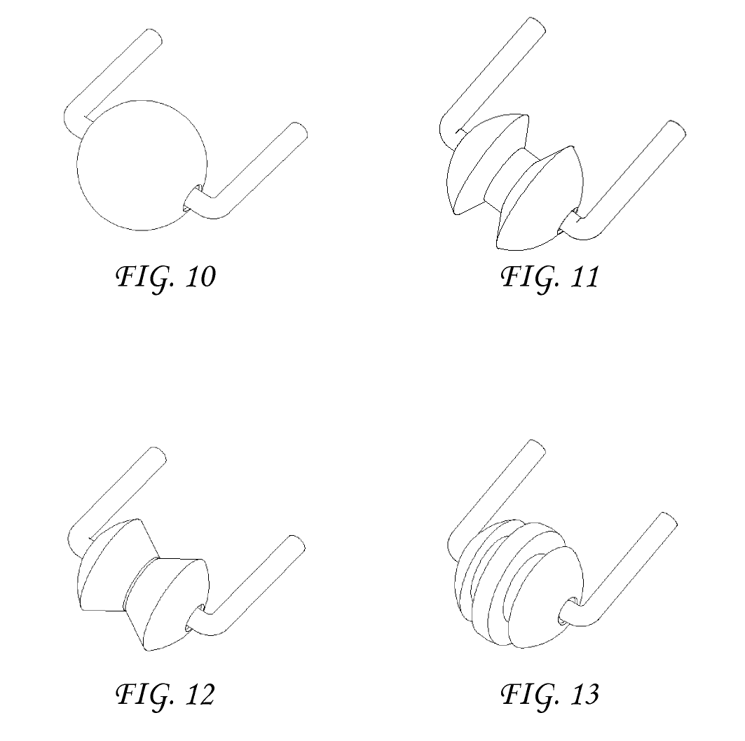

FIG. 4 is a “V” grooved slider electrode head. FIG. 5 is a plain roller bar electrode head. FIG. 6 is a square grooved roller bar electrode head. FIG. 7 is an axial grooved roller bar electrode head. FIG. 8 is a “V” grooved roller bar electrode head. FIG. 9 is an axial cum “V” grooved electrode head. FIG. 10 is a plain roller ball electrode head. FIG. 11 is a dumbbell roller ball electrode head. FIG. 12 is a “V” grooved roller ball electrode head. FIG. 13 is a square grooved roller ball electrode head.

Any of these electrodes can be used with resectoscopes for various procedures such as ablation of soft tissue in genitourinary tract, bladder and prostate. Resectoscopes generally have four elements, outer sheath, working element, electrode and telescope. The electrodes can be fixed in the working element and slide on the telescope tube. An electrosurgical radio frequency energy is applied to the electrode; the energized electrode is moved across the tissue being treated, performing the ablation. Peak voltage of the electrosurgical energy is in the range of 225 to 250 volts at power level of 60 to 250 watts.

The dimensions of the various elements of the electrode assembly can vary significantly depending upon application. The following approximate dimensions are considered exemplary. The tungsten wire diameter can be 0.35 mm (range of ˜0.25-0.4 mm). The tungsten wire length can be 360 mm (range of ˜300-500 mm). An electrode head can have a diameter of 3.2 mm (range of ˜3-5 mm). Electrode thickness can be 1.7 mm (range ˜1.5-4 mm). The bore hole diameter should be slightly larger than the diameter of the wire used.

Although the invention has been described with reference to specific embodiments, this description is not meant to be construed in a limiting sense. Various modifications of the disclosed embodiments, as well as alternative embodiments of the invention, will become apparent to persons skilled in the art upon reference to the description of the invention. It is therefore contemplated that such modifications can be made without departing from the spirit or scope of the present invention as defined in the appended claims.

What is claimed is:

1. A resection head for electrosurgical vaporization comprising:
at least one electrically conductive wire having a distal portion; and
an electrode attached to said distal portion of said wire wherein said electrode is formed of a heavy metal alloy material.

2. The resection head of claim 1 wherein the heavy metal alloy material is a tungsten alloy material.

3. The resection head of claim 2 wherein the tungsten alloy material has a composition which includes at least two of nickel, copper or iron, and is 85% to 96% by weight tungsten.

4. The resection head of claim 1 wherein said electrode has a bore hole formed therein, and said electrically conductive wire has a loop which passes through the bore hole.

5. The resection head of claim 1 wherein said electrode has a front side for engaging tissue to be vaporized and has a planar back side generally opposite said front side, said planar back side having a cavity formed therein, and said electrically conductive wire having two wire sections leading to said planar back side with an insulating material surrounding the wire sections, the insulating material extending from each of said wire sections into the cavity and abutting the heavy metal alloy material of said electrode.

6. The resection head of claim 1 wherein said electrically conductive wire is rigid with a loop formed at the distal portion, said electrode being attached to the loop, and said electrically conductive wire includes two wire sections emanating from the distal portion forming an angle in the range of 70° to 90°.

7. A resection head for electrosurgical vaporization comprising:
an electrode; and
at least one electrically conductive wire having a distal portion with a loop formed at said distal portion, said electrode being held in the loop, wherein said electrically conductive wire is rigid and includes two wire sections emanating from the distal portion forming an angle in the range of 70° to 90°.

8. The resection head of claim 7 wherein said electrode is formed of a tungsten heavy metal alloy material.

9. The resection head of claim 7 wherein said electrode has a bore hole formed therein, and the loop of said electrically conductive wire passes through the bore hole.

10. The resection head of claim 7 wherein said electrode has a front side for engaging tissue to be vaporized and has a planar back side generally opposite said front side, said planar back side having a cavity formed therein, said two wire sections leading to said planar back side with an insulating material surrounding said wire sections, the insulating material extending from each of said wire sections into the cavity and abutting said electrode.

11. A resection head for electrosurgical vaporization comprising:
an electrode have a bore hole formed therein; and
at least one electrically conductive wire having a loop which passes through the bore hole.

12. The resection head of claim 11 wherein said electrode has a front side for engaging tissue to be vaporized and has a planar back side generally opposite said front side, said planar back side having a cavity formed therein, and said electrically conductive wire having two wire sections leading to said planar back side with an insulating material surrounding the wire sections, the insulating material extending from each of said wire sections into the cavity and abutting said electrode.

13. The resection head of claim 11 wherein said electrode has a front side for engaging tissue to be vaporized and has a back side generally opposite said front side, with a cavity formed in said front side, the cavity being in fluid communication with the bore hole.

14. The resection head of claim 11 wherein said electrode is formed of a tungsten heavy metal alloy material.

15. The resection head of claim 11 wherein said electrically conductive wire is rigid, said electrode being held in the loop, and said electrically conductive wire includes two wire sections emanating from the distal portion forming an angle in the range of 70° to 90°.

16. A resection head for electrosurgical vaporization comprising:
an electrode formed of a tungsten heavy metal alloy material having a composition which includes at least two of nickel, copper or iron and is 85% to 96% by weight tungsten, said electrode having a bore hole formed therein, a front side for engaging tissue to be vaporized, and a planar back side generally opposite said front side, with a first cavity formed in said front side in fluid communication with the bore hole, and a second cavity formed in said planar back side; and
at least one electrically conductive wire having a distal portion with a loop which passes through the bore hole, said electrically conductive wire being rigid and including two wire sections leading to said planar back side forming an angle in the range of 70° to 90°, said two wire sections having an insulating material surrounding the wire sections, the insulating material extending from each of said wire sections into the second cavity and abutting the tungsten metal alloy material of said electrode.
